(19) United States
(12) Patent Application Publication
(10) Pub. No.: US 2023/0355115 A1
(43) Pub. Date: Nov. 9, 2023

(54) CUFF AND BLOOD PRESSURE MEASUREMENT DEVICE

(71) Applicant: OMRON HEALTHCARE Co., Ltd., Kyoto (JP)

(72) Inventor: Masaki HARADA, Kyoto (JP)

(21) Appl. No.: 18/355,071

(22) Filed: Jul. 19, 2023

Related U.S. Application Data
(63) Continuation of application No. PCT/JP2022/002598, filed on Jan. 25, 2022.

(30) Foreign Application Priority Data
Jan. 25, 2021 (JP) 2021-009600

(51) Int. Cl. A61B 5/022; A61B 5/00

(52) U.S. Cl. CPC A61B 5/02241; A61B 5/681; A61B 2562/0247

(57) ABSTRACT

A cuff and a blood pressure measurement device in which a sheet member constituting the cuff can easily be manufactured while suppressing the occurrence of a wrinkle affecting a blood pressure measurement result. A pressing cuff includes a plurality of air bags each configured by two sheet members long in one direction being fixed to each other at outer circumferential edges and layered with longitudinal directions aligned windably around a living body; a cutout portion provided in at least two sheet members of an air bag proximate to a wrist among the plurality of air bags and cut out from one end to the other end in a width direction; and a second fixing portion provided in a frame shape inside the outer circumferential edges of two sheet members facing each other of two adjacent air bags and fixing the two adjacent air bags to each other.

### CROSS-REFERENCE TO RELATED APPLICATIONS

This application is the U.S. national stage application filed pursuant to 35 U.S.C. 365(c) and 120 as a continuation of International Patent Application No. PCT/JP2022/002598, filed Jan. 25, 2022, which application claims priority to Japanese Patent Application No. 2021-009600, filed Jan. 25, 2021, which applications are incorporated herein by reference in their entireties.

### TECHNICAL FIELD

The present invention relates a cuff used in a blood pressure measurement device for measuring blood pressure, and a blood pressure measurement device.

### BACKGROUND ART

In recent years, blood pressure measurement devices used for measuring a blood pressure are being used as means to check health status at home, as well as in medical facilities. A blood pressure measurement device detects vibration of the artery wall to measure blood pressure by, for example, inflating and contracting a cuff wound around the upper arm or the wrist of a living body and detecting the pressure in the cuff by using a pressure sensor.

However, when the cuff is wound around the living body and inflated, a difference occurs between a circumference of an outer circumferential surface of the inflated cuff and a circumference of an inner circumferential surface of the inflated cuff, and thus wrinkles occur on a side of the cuff facing a living body. The number, position, depth, and the like of wrinkles formed in the cuff vary depending on a circumference and a shape of the living body around which the cuff is wound, the way of winding the cuff, and the like.

The wrinkle formed in the cuff divides the internal space of the cuff or causes loss of inflation pressure depending on the number, position, depth, and the like of the formed wrinkles, thereby causing a reduction in blood pressure measurement accuracy or variation in measurement results.

In view of this, a technique is known in which a groove is formed in the outer surface of the cuff facing the living body to generate a wrinkle that intersect the direction in which the cuff is wound around the living body, and the wrinkle is generated at the position of the groove, thereby controlling the position at which the wrinkle is generated (see Patent Document 1).

In addition, a technique is known in which an edge portion of a cuff is partially welded and a wrinkle is generated in the welded portion to control a position at which the wrinkle is generated (see Patent Document 2).

### PATENT LITERATURE

### Citation List

Patent Document 1: JP 2019-118407 A

Patent Document 2: JP 2019-5561 A

### SUMMARY OF INVENTION

### Technical Problem

In the cuff having the groove on the outer surface facing the living body, a sheet member constituting the cuff is to be subjected to primary processing such as profile extrusion, injection molding, or embossing for forming the groove. Thus, the number of manufacturing steps for the sheet member constituting the cuff is increased.

In the technique in which the edge portion of the cuff is partially welded and the wrinkle is generated in the welded portion to control the position at which the wrinkle is generated, the welded portion may inhibit the inflation of the cuff, resulting in insufficient compression force, and as a result, reduction in the accuracy of the blood pressure measurement.

Thus, an object of the present invention is to provide a cuff and a blood pressure measurement device that can reduce the number of manufacturing steps for a sheet member constituting the cuff while controlling the generation of the wrinkle.

### Solution to Problem

According to an aspect, there is provided a cuff including a plurality of bag-like structures layered with longitudinal directions aligned windably around a living body, each of the plurality of bag-like structures being configured by two sheet members long in one direction being fixed to each other at outer circumferential edges; a cutout portion provided in at least two sheet members of a bag-like structure proximate to a living body among the plurality of bag-like structures, the cutout portion being cut out from one end toward the other end in a width direction; and a fixing portion provided in a frame shape inside the outer circumferential edges of a sheet member of the two sheet members and a sheet member of the two sheet members facing each other of the corresponding two adjacent bag-like structures of the plurality of bag-like structures, the fixing portion fixing the two adjacent bag-like structures to each other.

Here, the cuff includes a bag-like structure which is wound around an upper arm, a wrist, or the like of a living body when a blood pressure is measured and is inflated by being supplied with a fluid. The cuff is inflated by the supplied fluid. Here, the fluid includes a liquid and air. When the supplied fluid is air, the bag-like structure is, for example, an air bag which is inflated by air.

According to this aspect, when the cuff is inflated and an inner-outer circumferential difference is generated between the inner circumferential surface and the outer circumferential surface of the cuff, the cutout portion serves as a starting point where a wrinkle is generated.

The wrinkle includes a bulge toward the living body and a recess toward the outer circumferential surface, which are generated in a part of the inner circumferential surface of the bag-like structure, and a fold generated in the inner circumferential surface of the bag-like structure, when the bag-like structure is inflated in a state of being wound around the living body and a difference (inner-outer circumferential difference) in circumference is generated between the outer circumferential surface and the inner circumferential surface of the bag-like structure.

Although the wrinkle is generated due to the inner-outer circumferential difference of the bag-like structure, the position where the wrinkle is generated can be controlled by the cutout portion. In addition, it is possible to control the number and the depth of the wrinkles by setting the cutout portion depending on the condition when the blood pressure measurement device is used, the thickness of the wrist of the user who assumes the use of the blood pressure measurement device, and the like. Thus, when the blood pressure is measured, it is possible to suppress a variation from occurring in the blood pressure measurement result and to improve the accuracy of the blood pressure measurement result.

Furthermore, the bag-like structure is configured by fixing two sheet members at their outer circumferential edges. Two adjacent bag-like structures are fixed by a frame-like fixing portion on an inner side of the outer circumferential edges of two sheet members facing each other. Because the two adjacent bag-like structures are fixed by the fixing portion at a position inside the cutout portion, the cutout portion does not hinder the inflation of the bag-like structures.

For example, in the case of a configuration in which a part of each of the plurality of bag-like structures is welded in the layering direction and a cutout portion is formed in a part of the welded region, the welded region affects the inflation of the plurality of bag-like structures, that is, affects the inflation of the cuff

Furthermore, by forming the cutout portion in a shape that is cut out from one end toward the other end in a width direction of the two sheet members, wrinkling can be controlled with a simple configuration. Thus, it is not necessary to perform primary processing for forming a groove that causes the wrinkle in the sheet member constituting the bag-like structure. Thus, the number of manufacturing steps of the sheet member constituting the bag-like structure can be reduced.

In the cuff according to the aspect, there is provided the cuff in which the cutout portion is formed avoiding a position facing an artery in a state where the cuff is wound around the living body.

According to this aspect, the cutout portion is disposed at a position avoiding a position facing the artery when the blood pressure measurement device is worn. Thus, it is possible to suppress the generation of the wrinkle at the position of the cuff facing the artery, and even if the wrinkle is generated at this position when the cuff is inflated, it is possible to make the wrinkle shallower than the wrinkle generated by the cutout portion.

In the cuff according to the aspect, there is provided the cuff in which a plurality of the cutout portions are provided.

According to this aspect, it is possible to control the position where the wrinkle is generated more suitably.

According to an aspect, there is provided a blood pressure measurement device including any one of the cuffs described above; a pump that supplies fluid to the cuff; a pressure sensor that detects a pressure in the cuff; and a body attached to the cuff and including a control board being built into the body.

According to this aspect, when the cuff is inflated and an inner-outer circumferential difference is generated between the inner circumferential surface and the outer circumferential surface of the cuff, the cutout portion serves as a starting point where the wrinkle is generated.

Although the wrinkle is generated due to the inner-outer circumferential difference of the bag-like structure, the position where the wrinkle is generated can be controlled by the cutout portion. In addition, it is possible to control the number and the depth of the wrinkles by setting the cutout portion depending on the condition when the blood pressure measurement device is used, the thickness of the wrist of the user who assumes the use of the blood pressure measurement device, and the like. Thus, when the blood pressure is measured, it is possible to suppress a variation from occurring in the blood pressure measurement result and to improve the accuracy of the blood pressure measurement result.

Furthermore, the bag-like structure is configured by fixing two sheet members at their outer circumferential edges. Two adjacent bag-like structures are fixed by a frame-like fixing portion on an inner side of the outer circumferential edges of two sheet members facing each other. Because the two adjacent bag-like structures are fixed by the fixing portion at a position inside the cutout portion, the cutout portion does not hinder the inflation of the bag-like structures.

For example, in the case of a configuration in which a part of each of the plurality of bag-like structures is welded in the layering direction and a cutout portion is formed in a part of the welded region, the welded region affects the inflation of the plurality of bag-like structures, that is, affects the inflation of the cuff.

Furthermore, by forming the cutout portion in a shape that is cut out from one end toward the other end in a width direction of the two sheet members, wrinkling can be controlled with a simple configuration. Thus, it is not necessary to perform primary processing for forming a groove that causes the wrinkle in the sheet member constituting the bag-like structure. Thus, the number of manufacturing steps of the sheet member constituting the bag-like structure can be reduced.

In the blood pressure measurement device according to the aspect, there is provided the blood pressure measurement device in which the cutout portion is formed avoiding a position facing an artery in a state where the blood pressure measurement device is attached to the living body.

According to this aspect, the cutout portion is disposed at a position avoiding a position facing the artery when the blood pressure measurement device is worn.

Thus, it is possible to suppress the generation of the wrinkle at the position of the cuff facing the artery, and even if the wrinkle is generated at this position when the cuff is inflated, it is possible to make the wrinkle shallower than the wrinkle generated by the cutout portion.

According to the blood pressure measurement device of the aspect, there is provided the blood pressure measurement device in which a plurality of the cutout portions are provided.

According to this aspect, it is possible to control the position where the wrinkle is generated more suitably.

According to the blood pressure measurement device of the aspect, there is provided the blood pressure measurement device including a flow path fluidly communicating with the cuff, in which the flow path passes through the cutout portion.

According to this aspect, the flow path can be disposed in the cuff from one side to the other side of the cuff

According to the blood pressure measurement device of the aspect, there is provided the blood pressure measurement device including electric wiring electrically connected to the control board, in which the electric wiring passes through the cutout portion.

According to this aspect, the electric wiring can be disposed on the cuff from one side to the other side of the cuff.

### Advantageous Effects of Invention

According to the present invention, it is possible to provide a cuff and a blood pressure measurement device in which the number of manufacturing steps of a sheet member constituting the cuff can be reduced while suppressing the occurrence of a wrinkle that affects a blood pressure measurement result.

### BRIEF DESCRIPTION OF DRAWINGS

FIG. 1 is a perspective view illustrating a configuration of a blood pressure measurement device according to an embodiment of the present invention.

FIG. 2 is a cross-sectional view illustrating a configuration of a cuff structure used in the blood pressure measurement device.

FIG. 3 is a plan view illustrating a configuration of the cuff structure.

FIG. 4 is a side view illustrating a configuration of a pressing cuff used in the cuff structure.

FIG. 5 is a side view schematically illustrating a configuration of the pressing cuff at the time of inflation.

FIG. 6 is a plan view illustrating a configuration of a first modification of the pressing cuff.

FIG. 7 is a plan view illustrating a configuration of a second modification of the pressing cuff used in the blood pressure measurement device according to the present embodiment.

FIG. 8 is a plan view illustrating a pressing cuff and electric wiring used in a blood pressure measurement device according to a modification of the present embodiment.

### DESCRIPTION OF EMBODIMENTS

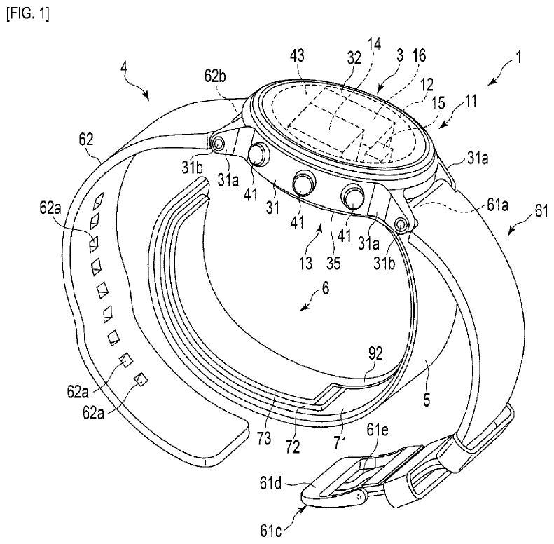

Hereinafter, an example of a blood pressure measurement device 1 according to an embodiment of the present invention will be described with reference to FIGS. 1 to 5.

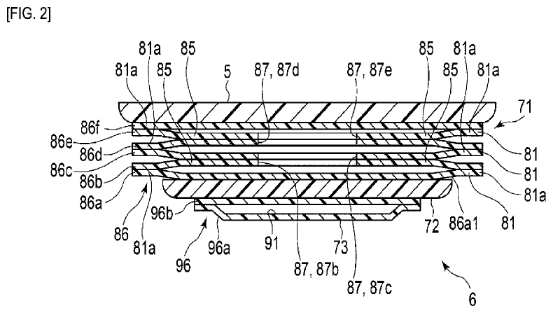

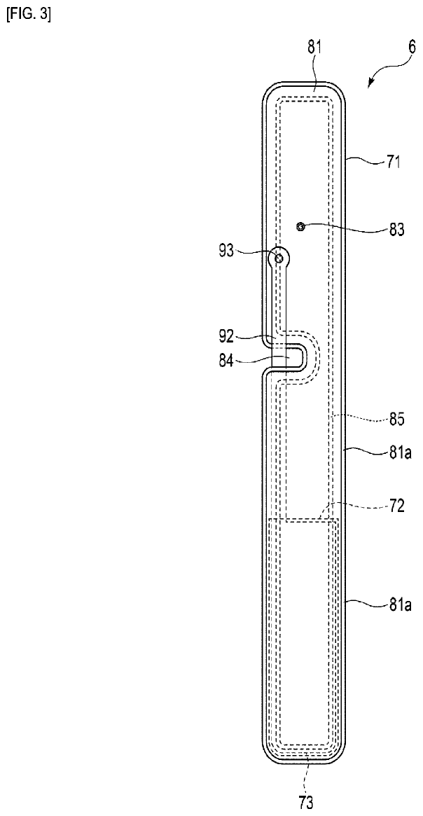

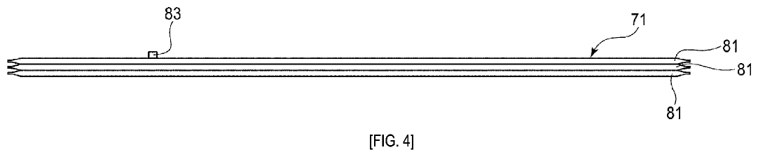

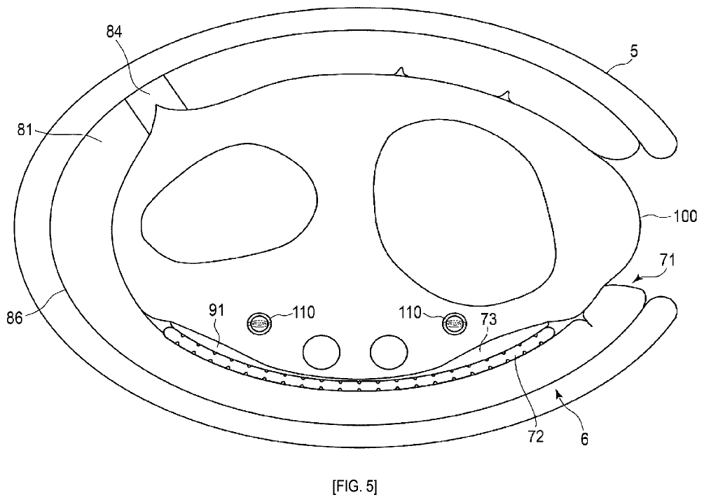

FIG. 1 is a perspective view illustrating the configuration of the blood pressure measurement device 1. FIG. 2 is a cross-sectional view illustrating the configuration of a cuff structure 6 used in the blood pressure measurement device 1. FIG. 3 is a plan view illustrating a configuration of the cuff structure 6. FIG. 4 is a side view illustrating a configuration of a pressing cuff 71 used in the cuff structure 6. FIG. 5 is a side view schematically illustrating the configuration of the pressing cuff 71 at the time of inflation.

The blood pressure measurement device 1 is an electronic blood pressure measurement device worn on a living body. In the present embodiment, the blood pressure measurement device 1 is an electronic blood pressure measurement device having a form of a wearable device to be worn on a wrist 100 and having a form of measuring blood pressure from an artery 110.

As illustrated in FIGS. 1 and 2, the blood pressure measurement device 1 includes, for example, a device body 3, a belt 4, a curler 5, and the cuff structure 6.

As illustrated in FIG. 1, the device body 3 includes, for example, a case 11, a display unit 12, and an operation portion 13. Furthermore, the device body 3 includes a pump 14 for inflating the cuff structure 6, a pressure sensor 15, a flow path portion for fluidly connecting the pump 14 and the cuff structure 6, and a control board 16. For example, one or a plurality of on-off valves and the pressure sensor 15 that detects the pressure in the cuff of the cuff structure 6 are connected to the flow path portion.

The case 11 includes, for example, an outer case 31 having a cylindrical shape, a windshield 32 that covers an upper opening of the outer case 31, and a back lid 35 that covers a lower portion of the inside of the outer case 31.

The outer case 31 is formed in, for example, a cylindrical shape. The outer case 31 includes pairs of lugs 31a provided at respective symmetrical positions in the circumferential direction of an outer circumferential surface, and spring rods 31b each provided between the corresponding two pairs of lugs 31a. The windshield 32 is, for example, a circular glass plate. The back lid 35 covers an end portion of the outer case 31 on the wrist side. The back lid 35 is fixed to, for example, an end portion on the wrist side of the outer case 31.

The display unit 12 is disposed at a position of the outer case 31 facing the windshield 32. The display unit 12 is electrically connected to the control board 16. The display unit 12 is, for example, a liquid crystal display or an organic electroluminescence display. The display unit 12 displays various types of information including the date and time, measurement results of blood pressure values such as the systolic blood pressure and diastolic blood pressure, heart rate.

The operation portion 13 is configured to be able to input a command from a user. For example, the operation portion 13 includes a plurality of buttons 41 provided on the case 11, a sensor that detects operation of each of the buttons 41, and a touch panel 43 provided on the display unit 12 or the windshield 32. The operation portion 13 is operated by a user to convert a command into an electric signal and output the electric signal to the control board 16.

The pump 14 is, for example, a piezoelectric pump. The pump 14 compresses air and supplies the compressed air to the cuff structure 6 via the flow path portion. The pump 14 is electrically connected to the control board 16.

The belt 4 includes a first belt 61 provided on one pair of lugs 31a and the spring rod 31b, and a second belt 62 provided on the other pair of lugs 31a and the spring rod 31b.

The first belt 61 is a so-called parent, is formed in a belt shape, and has a buckle 61c. The first belt 61 is rotatably held by the outer case 31. The buckle 61c includes a frame body 61d having a rectangular frame shape and a prong 61e rotatably attached to the frame body 61d.

The second belt 62 is referred to as a so-called blade tip and is configured in a band-like shape having a width at which the second belt 62 can be inserted into the frame body 61d. In addition, the second belt 62 includes a plurality of small holes 62a into which the prong 61e is inserted. The second belt 62 is rotatably held by the outer case 31.

In the belt 4 as described above, the second belt 62 is inserted into the frame body 61d and the prong 61e is inserted into the small hole 62a to integrally connect the first belt 61 and the second belt 62, and the belt 4 comes to have an annular shape extending in such a manner as to follow along the circumferential direction of the wrist 100 together with the outer case 31.

Because the belt 4 becomes annular, the belt 4 presses the curler 5 and the cuff structure 6 toward the wrist 100 when the cuff structure 6 is inflated.

The curler 5 is made of, for example, a resin material and is formed in a band-like shape curved along the circumferential direction of the wrist. One end of the curler 5 is fixed to the wrist 100 side of the device body 3, and the other end is configured to be close to the device body 3. In the present embodiment, as illustrated in FIG. 1, the curler 5 may be fixed to the outer surface of the back lid 35, one end of the curler 5 may protrude from a portion of the back lid 35 proximate to one pair of lugs 31a, the curler 5 may protrude toward a portion of the back lid 35 proximate to the other pair of lugs 31a from the one end of the curler 5 to the other end of the curler 5, and the other end of the curler 5 may extend to a position adjacent to the one end of the curler 5.

The curler 5 has hardness appropriate to provide flexibility and shape retainability. Here, “flexibility” means that the shape is deformed in the radial direction when an external force is applied to the curler 5 and that, for example, the shape of the curler 5 in a side view is deformed so as to come close to the wrist, follow the shape of the wrist, or imitate the shape of the wrist when the curler 5 is pressed by belt 4. In addition, “shape retainability” means that the curler 5 can maintain a shape formed in advance when no external force is applied, that is, the curler 5 can maintain a shape curved along the circumferential direction of the wrist 100 in the present embodiment. In the present description, a surface of the curler 5 facing the wrist is referred to as an inner surface of the curler 5. The curler 5 holds the cuff structure 6 along the inner surface shape of the curler 5.

The cuff structure 6 includes one or a plurality of cuffs and is connected to the pump 14 via a flow path portion. The cuff structure 6 presses the wrist 100 when the cuff is inflated by the fluid from the pump 14.

As illustrated in FIG. 2, the cuff structure 6 includes, for example, the pressing cuff 71, a back plate 72, and a sensing cuff 73. The cuff structure 6 is integrally formed by layering the pressing cuff 71, the back plate 72, and the sensing cuff 73. The cuff structure 6 is fixed to the inner surface of the curler 5.

As illustrated in FIGS. 2 and 3, the pressing cuff 71 is provided with a plurality of air bags 81 that are layered with longitudinal directions aligned, a connection portion 83, and a second fixing portion 85 that fixes two adjacent air bags 81 among the plurality of air bags 81.

The pressing cuff 71 is fixed to the curler 5 by attaching the outer air bag 81 to the inner surface of the curler 5 with a double-sided tape, an adhesive, or the like.

The pressing cuff 71 is fluidly connected to the pump 14 by the connection portion 83 being connected to the flow path portion. The pressing cuff 71 is inflated to press the back plate 72 and the sensing cuff 73 toward the wrist. The pressing cuff 71 has a length extending from the side of the wrist 100 same as the back of the hand to the side of the wrist 100 same as the palm when the blood pressure measurement device 1 is worn on the wrist.

The plurality of air bags 81 are, for example, three layers of air bags 81. Here, the air bag 81 has a bag-like structure. In the present embodiment, because the blood pressure measurement device 1 is configured to use air by the pump 14, the description will be made using an air bag. However, when a fluid other than air is used, the bag-like structure may be a fluid bag.

The air bag 81 is formed in a shape that is long in one direction by overlapping two sheet members 86 that are long in one direction and welding the outer circumferential edges by heat.

For example, the air bag 81 is provided with a first fixing portion 81a that fixes the outer circumferential edges of the two sheet members 86. The first fixing portion 81a is, for example, a welded portion where the two sheet members 86 are thermally welded. The outer circumferential edges of the two sheet members 86 on which the first fixing portion 81a is provided also include the outer circumferential edge of the cutout portion 84 formed in the sheet member 86.

In addition, the plurality of air bags 81 communicate with each other so that air can move. For example, an opening 87 is formed in two sheet members 86 facing each other of two adjacent air bags 81 among the plurality of air bags 81. Thus, the two adjacent air bags 81 communicate with each other through the opening 87.

The cutout portion 84 is formed in a shape that is cut out from one end toward the other end in the width direction of the air bag 81, that is, a shape that is recessed so that the width becomes narrower from one end toward the other end in the width direction of the air bag 81. At the position where the cutout portion 84 is formed, when the pressing cuff 71 is inflated to press the wrist, a wrinkle is generated on the surface of the air bag 81 on the wrist 100 side. The wrinkle is a bulge toward the wrist 100 side or a recess toward the outer circumferential surface side, which is generated in a part of the inner circumferential surface, or a fold generated in the inner circumferential surface of the air bag 81 such that the inner circumferential surface is positioned toward the outer circumferential surface, when the air bag 81 is inflated in a state of being wound around the wrist 100 and a difference (inner-outer circumferential difference) in circumference between the outer circumferential surface and the inner circumferential surface of the air bag 81 is generated.

For example, the cutout portion 84 is formed at the same position in all of the plurality of air bags 81. In the present embodiment, the cutout portion 84 is formed at the same position in the longitudinal direction of each of the three air bags 81. The cutout portion 84 is formed at a position of the air bag 81 that does not affect the blood pressure measurement. Here, the position that does not affect the blood pressure measurement is, for example, a position avoiding a region of the pressing cuff 71 facing the artery 110 when the blood pressure measurement device 1 is worn on the wrist 100. In the present embodiment, as illustrated in FIG. 3, the cutout portion 84 is formed at a position avoiding the sensing cuff 73.

One or a plurality of the cutout portions 84 are formed in one air bag 81. In the present embodiment, one cutout portion 84 is formed in one air bag 81.

Furthermore, around the cutout portion 84, the first fixing portion 81a is formed along the cutout portion 84. That is, around the cutout portion 84, the first fixing portion 81a has a shape recessed from one end to the other side in the width direction of the air bag 81.

The cutout portion 84 has a predetermined width in the width direction and the longitudinal direction of the sheet member 86. The predetermined width is appropriately set in accordance with the number and depth of the wrinkles to be generated. The dimension of the cutout portion 84 along the width direction of the air bag 81 and the dimension of the cutout portion 84 along the longitudinal direction of the air bag 81 are appropriately selected as long as the cutout portion 84 is configured to be able to generate a predetermined wrinkle. Here, the predetermined wrinkle refers to a wrinkle having a depth that does not divide the internal space of the air bag 81 when the pressing cuff 71 that curves imitating the shape of the wrist 100 in the circumferential direction is inflated. Because the cutout portion 84 has a predetermined width such that the wrinkle to be generated do not divide the internal space of the air bag 81, one side and the other side in the longitudinal direction of the air bag 81 with the cutout portion 84 interposed therebetween are maintained in a communicating state.

In addition, the cutout portion 84 has, for example, a dimension along the width direction of the pressing cuff 71 and a dimension along the longitudinal direction of the pressing cuff 71 through which a flow path body 92 of the sensing cuff 73 can pass.

The second fixing portion 85 fixes two of the plurality of adjacent layered air bags 81. The second fixing portion 85 is formed in a frame shape inside the first fixing portion 81a between the two air bags 81. The second fixing portion 85 is a welded portion formed by, for example, thermally welding the sheet members 86 facing each other of the adjacent air bags 81. For example, the second fixing portion 85 is formed in a frame shape surrounding the opening 87 formed in the sheet members of the two adjacent air bags 81.

The connection portion 83 is connected to the flow path portion. For example, the connection portion 83 is provided in one air bag 81 of the plurality of air bags 81 proximate to the curler 5. The connection portion 83 is, for example, a nipple.

As illustrated in FIG. 2, the three-layer air bags 81 include, from the wrist 100 side, a first sheet member 86a, a second sheet member 86b constituting the first layer air bag 81 together with the first sheet member 86a, a third sheet member 86c integrally bonded to the second sheet member 86b, a fourth sheet member 86d constituting the second layer air bag 81 together with the third sheet member 86c, a fifth sheet member 86e integrally bonded to the fourth sheet member 86d, a sixth sheet member 86f constituting the third layer air bag 81 together with the fifth sheet member 86e. Each of the sheet members 86a, 86b, 86c, 86d, 86e, and 86f has a cutout constituting the cutout portion 84.

The first sheet member 86a and the second sheet member 86b are fixed at their outer circumferential edges by the first fixing portion 81a, thereby forming the air bag 81. The third sheet member 86c and the fourth sheet member 86d are fixed at their outer circumferential edges by the first fixing portion 81a, thereby forming the air bag 81. The fifth sheet member 86e and the sixth sheet member 86f are fixed at their outer circumferential edges by the first fixing portion 81a, thereby forming the air bag.

An adhesive layer or a double-sided tape is provided on the outer surface of the sixth sheet member 86f facing the curler 5, and the sixth sheet member 86f is attached to the curler 5 by the adhesive layer or the double-sided tape.

The second sheet member 86b and the third sheet member 86c are disposed facing each other and respectively have openings 87b and 87c through which the two air bags 81 communicate with each other. The second sheet member 86b and the third sheet member 86c are fixed by the second fixing portion 85 at a position inside the first fixing portion 81a of the outer circumferential edge forming the air bag 81 and at a position outside the openings 87b and 87c.

The fourth sheet member 86d and the fifth sheet member 86e are disposed facing each other and respectively have openings 87d and 87e through which the two air bags 81 communicate with each other. The fourth sheet member 86d and the fifth sheet member 86e are fixed by the second fixing portion 85 at a position inside the first fixing portion 81a of the outer circumferential edge forming the air bag 81 and at a position outside the openings 87d and 87e.

As illustrated in FIG. 2, the back plate 72 is attached to an outer surface 86a1 of the first sheet member 86a of the pressing cuff 71 by an adhesive layer, a double-sided tape, or the like. The back plate 72 is formed of a resin material and is formed in a plate shape. The back plate 72 is made of polypropylene, for example, and is formed into a plate shape having a thickness of approximately 1 mm. The back plate 72 has shape followability.

Here, “shape followability” refers to a characteristic that the back plate 72 can be deformed to imitate the shape of a contacted portion of the wrist 100 on which the back plate 72 is disposed. Note that the contacted portion of the wrist 100 refers to a region in contact with the back plate 72, and the contact here includes both direct contact and indirect contact.

The sensing cuff 73 is fixed to a surface of the back plate 72 facing the wrist 100. The sensing cuff 73 comes into direct contact with a region of the wrist 100 where the artery 110 is present. The sensing cuff 73 is formed in the same shape as that of the back plate 72 or a shape that is smaller than that of the back plate 72, in the longitudinal direction and the width direction of the back plate 72. The sensing cuff 73 compresses the region of the wrist on the side same as the palm where the artery is present by being inflated. The sensing cuff 73 is pressed toward the wrist 100 via the back plate 72 by the inflated pressing cuff 71.

As illustrated in FIGS. 2 and 3, the sensing cuff 73 includes one air bag 91 and a flow path body 92. In the sensing cuff 73, one surface of the air bag 91 is fixed to the back plate 72. For example, the sensing cuff 73 is attached to the surface of the back plate 72 proximate to the living body by a double-sided tape, an adhesive layer, or the like.

Here, the air bag 91 is a bag-like structure. In the present embodiment, because the blood pressure measurement device 1 is configured to use air by the pump 14, the description will be made using an air bag. However, when a fluid other than air is used, the bag-like structure may be a fluid bag.

The air bag 91 is formed in a shape that is long in one direction by overlapping two sheet members 96 that are long in one direction and welding their outer circumferential edges by heat.

The flow path body 92 is integrally provided at a part of one edge of the air bag 91 in the longitudinal direction. The flow path body 92 is provided at an end portion close to the device body 3 in the longitudinal direction of the air bag 91. In addition, the flow path body 92 is formed in a shape which is long in one direction with a width smaller than the dimension of the air bag 91 in the width direction. The flow path body 92 includes the connection portion 93 on its tip. The flow path body 92 is connected to the flow path portion via the connection portion 93 and forms a flow path between the flow path portion and the air bag 91.

As illustrated in FIG. 3, for example, a part of the flow path body 92 is disposed in the cutout portion 84. That is, in order to cause the sensing cuff 73 disposed on the surface of the pressing cuff 71 on the side close to the wrist 100 to communicate with the flow path portion on the opposite side of the pressing cuff 71, the flow path body 92 is moved from the surface of the pressing cuff 71 on the side close to the wrist 100 to the surface on the opposite side through the cutout portion 84.

The connection portion 93 is, for example, a nipple. The connection portion 93 is provided at the tip of the flow path body 92. The connection portion 93 is connected to the flow path portion and is connected to the pump 14 via the flow path portion.

As illustrated in FIG. 2, the sensing cuff 73 is provided with a seventh sheet member 96a and an eighth sheet member 96b from the wrist 100 side. For example, the seventh sheet member 96a and the eighth sheet member 96b are configured in a shape that allows to configure the air bag 91 and the flow path body 92.

The air bag 91 and the flow path body 92 are constituted by the seventh sheet member 96a and the eighth sheet member 96b being disposed facing each other, welded by heat along the circumferential edge shapes of the air bag 91 and the flow path body 92 so that the air bag 91 fluidly communicates with the flow path body 92, and cut in a predetermined shape.

The sheet members 86 and 96 forming the pressing cuff 71 and the sensing cuff 73 are made of a thermoplastic elastomer. Examples of thermoplastic elastomer constituting the sheet members 86 and 96 include thermoplastic polyurethane-based resin (hereinafter referred to as TPU), polyvinyl chloride resin, ethylene-vinyl acetate resin, thermoplastic polystyrene-based resin, thermoplastic polyolefin resin, thermoplastic polyester-based resin, and thermoplastic polyamide resin. As the thermoplastic elastomer, TPU is preferably used. The sheet member may have a single-layer structure or a multi-layer structure.

The sheet members 86 and 96 are not limited to a thermoplastic elastomer and may be a thermosetting elastomer such as silicone or may be a combination of a thermoplastic elastomer (for example, TPU) and a thermosetting elastomer (for example, silicone).

For the sheet members 86 and 96, a molding method such as T-die extrusion molding, injection molding, blow molding, or calendar molding is used when a thermoplastic elastomer is used, and a molding method such as metal mold cast molding is used when a thermosetting elastomer is used.

For example, the sheet members 86 and 96 are molded by each molding method, then bonded by adhesion, welding, or the like, and then sized into a predetermined shape, thereby forming the air bags 81 and 91.

The cutout constituting the cutout portion 84 of the sheet member 86 can be formed, for example, at the time of sizing. As a bonding method, a high-frequency welder or laser welding is used when a thermoplastic elastomer is used, and a molecular adhesive is used when a thermosetting elastomer is used.

In the pressing cuff 71 configured as described above, as illustrated in FIG. 5, in a state where the blood pressure measurement device 1 is worn on the wrist 100 and the pressing cuff 71 is sufficiently inflated to measure the blood pressure, a deep wrinkle is generated by the cutout portion 84. Wrinkles are also generated at positions where the cutout portion 84 is not provided but are shallower than the wrinkle generated by the cutout portion 84. In FIG. 5, for convenience, the plurality of air bags 81 constituting the pressing cuff 71 are collectively illustrated as one air bag 81.

In the blood pressure measurement device 1 according to an embodiment configured as described above, the pressing cuff 71 has the cutout portion 84 that causes a wrinkle. The two sheet members 86 constituting the air bag 81 of the pressing cuff 71 are fixed by the first fixing portion 81a at their outer circumferential edges including the cutout constituting the cutout portion 84. The two sheet members 86 facing each other of the two adjacent air bags 81 are fixed by the second fixing portion 85 inside the first fixing portion 81a. That is, the two adjacent air bags 81 are fixed by the second fixing portion 85 inside the cutout portion 84.

When the air bag 81 is inflated, portions of the two sheet members 86 constituting the air bag 81 inside the first fixing portion 81a are moved or deformed so as to be relatively separated from each other and thus the air bag 81 inflates, but the two sheet members 86 are fixed by the first fixing portion 81a at their outer circumferential edges including the cutout, so that the cutout portion 84 does not hinder the inflation of the air bag 81. Furthermore, the two sheet members 86 facing each other of the two adjacent air bags 81 are fixed by the second fixing portion 85 inside the cutout portion 84, so that the cutout portion 84 does not hinder the inflation of the two adjacent air bags 81. Accordingly, the cutout portion 84 does not affect the inflation of the pressing cuff 71.

For example, in the case of a configuration in which a part of each of the plurality of air bags 81 is welded in the layering direction and the cutout is formed in the welded region, the welded region affects the inflation of the plurality of air bags 81. That is, the inflation of the pressing cuff 71 is affected.

However, because the two adjacent air bags 81 are fixed by the second fixing portion 85 inside the cutout portion 84, the cutout portion 84 does not affect the inflation of the other air bags 81.

Furthermore, when the pressing cuff 71 of the cuff structure 6 provided on the inner surface of the curler 5 is inflated, the cutout portion 84 can generate a wrinkle at the position where the cutout portion 84 of the pressing cuff 71 is provided. Thus, the blood pressure measurement device 1 can improve the accuracy of the measured blood pressure measurement result.

Hereinafter, this effect will be described in detail. Because the curler 5 provided in the blood pressure measurement device 1 has a shape along the circumferential direction of the wrist 100, the pressing cuff 71 has a shape curved at a predetermined curvature. Thus, because the curvature radii of the inner circumferential surface and the outer circumferential surface of the pressing cuff 71 are different, the circumference of the inner circumferential surface and the circumference of the outer circumferential surface of the inflated pressing cuff 71 are different, and the inner-outer circumferential difference occurs. Due to the inner-outer circumferential difference, wrinkles are generated in a part of the inner circumferential surfaces of the plurality of air bags 81 of the pressing cuff 71. A part of the wrinkles may be a fold generated in the air bag 81 such that a part of the inner circumferential surface of the air bag 81 is positioned toward the outer circumferential surface.

Such wrinkles may divide the internal space of the pressing cuff 71 or may cause the inflation pressure to be lost, depending on the position and depth of the wrinkles. Furthermore, because the portion where the wrinkle is generated does not swell toward the wrist 100, the pressing force cannot be efficiently transmitted to the wrist 100 and the sensing cuff 73, and the pressure distribution on the surface of the pressing cuff 71 becomes uneven. Thus, there is a concern that the wrinkle generated on the inner circumferential surface of the pressing cuff 71 may cause an influence on a blood pressure measurement result such as a decrease in blood pressure measurement accuracy and a variation in measurement results.

However, the present embodiment has a configuration in which the cutout portion 84 serving as the starting point of the generation of the wrinkle is provided in the pressing cuff 71. Thus, by setting the position where the cutout portion 84 is provided, it is possible to control the position where the wrinkle is generated. Thus, when the blood pressure is measured, it is possible to suppress a variation from occurring in the blood pressure measurement result and to improve the accuracy of the blood pressure measurement result.

The wrinkle is likely to be deeper in a portion having a small radius of curvature. When the wrinkle becomes deep, the flow of air in the air bag 81 is impeded. Thus, it is preferable that cutout portion 84 is formed at a position avoiding a portion where the curvature radius of the curler facing the air bag 81 is smaller than the other portions.

In addition, it is preferable that the wrinkle is formed at a position having a small influence on the blood pressure measurement. For example, in the present embodiment, a configuration is adopted in which the cutout portion 84 is provided in a region of the air bag 81 where the sensing cuff 73 is not disposed, that is, in a region avoiding a position facing the artery, whereby it is possible to suppress the occurrence of the wrinkle in the region where the sensing cuff 73 is disposed. Accordingly, the pressing cuff 71 can more uniformly press the sensing cuff 73, and the sensing cuff 73 can suitably press the artery.

Furthermore, because the cutout portion 84 is constituted by the cutouts each formed in the two sheet members 86 constituting the corresponding one of the air bag 81 of the pressing cuff 71, it is not necessary to perform primary processing such as forming a groove that causes the wrinkle in the sheet member 86 constituting the air bag 81. Thus, the number of the manufacturing steps of the sheet member 86 can be reduced.

In addition, in the pressing cuff 71, because the position of the wrinkle generated in the pressing cuff 71 can be controlled by the cutout portion 84, even when the length of the pressing cuff 71 is set to a length extending from the side of the wrist 100 same as the back of the hand to the side of the wrist 100 same as the palm when the blood pressure measurement device 1 is worn on the wrist 100, it is possible to suppress the wrinkle which affects the blood pressure measurement from being generated in the pressing cuff 71. Accordingly, it is possible to suppress the number of cuffs included in the cuff structure 6 from increasing.

As described above, according to the blood pressure measurement device 1 of the present embodiment, by providing the cutout portion 84 in at least one of the plurality of air bags 81 of the pressing cuff 71, it is possible to generate the wrinkle at the position where the cutout portion 84 of the pressing cuff 71 is provided, and thus it is possible to improve the accuracy of the blood pressure measurement result.

Furthermore, because the primary processing for forming a groove or the like for generating the wrinkle in the sheet member 86 constituting the air bag 81 is unnecessary, the number of manufacturing steps of the sheet member 86 can be reduced.

In the above-described example, the configuration in which one cutout portion 84 is formed in one air bag 81 has been described, but the method of forming the cutout portion 84 is not limited thereto. In another example, a plurality of the cutout portions 84 may be formed in one air bag 81.

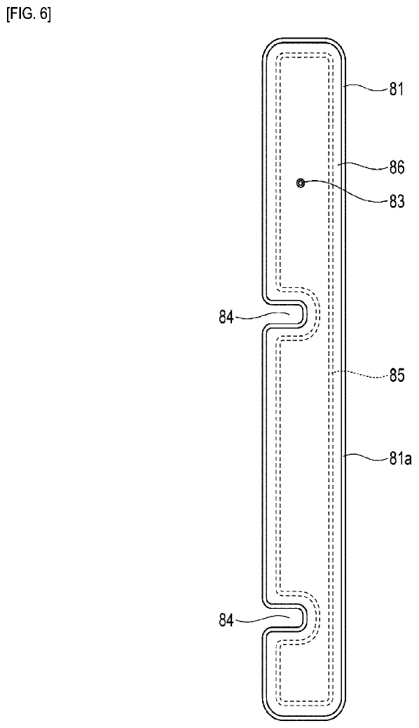

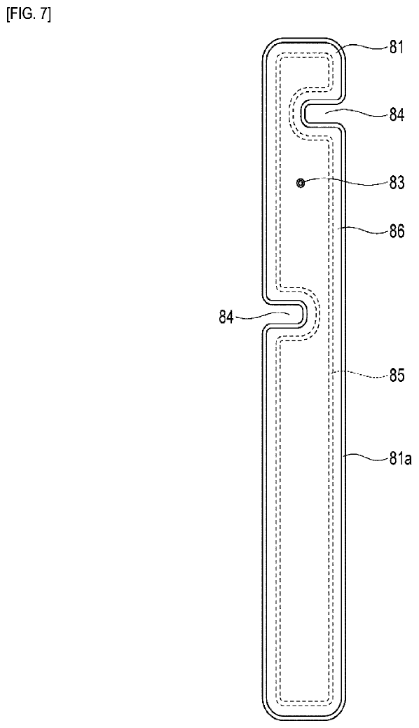

For example, as in a modification illustrated in FIG. 6, two cutout portions 84 may be formed at one end in the width direction of the air bag 81. Alternatively, as in a modification illustrated in FIG. 7, one cutout portion 84 may be formed at each of both ends in the width direction of the air bag 81. As illustrated in FIG. 7, the cutout portions 84 may be formed at different positions in the longitudinal direction of the pressing cuff 71 at both ends in the width direction of the pressing cuff 71. Alternatively, the cutout portions 84 may be formed at positions opposed to each other in the width direction at both ends of the pressing cuff 71 in the width direction. In addition, the shape of the cutout portion 84 can be appropriately set as long as the wrinkle can be controlled.

In addition, in the above-described example, the configuration in which the cutout portion 84 is provided in all of the plurality of air bags 81 included in the pressing cuff 71 has been described as an example, but the configuration is not limited thereto. In another example, the cutout portion 84 is provided in at least one of the plurality of air bags 81 of the pressing cuff 71. In this example, the cutout portion 84 is provided in at least one air bag 81 disposed proximate to the wrist 100 among the plurality of air bags 81. That is, the cutout portion 84 can be appropriately set as long as the wrinkle of the air bag 81 proximate to the living body can be controlled and each air bag 81 can be inflated.

In addition, in the above-described example, the configuration in which the flow path body 92 connecting the flow path portion in the device body 3 and the sensing cuff 73 passes through the cutout portion 84 has been described as an example, but a member connecting the device body 3 and the cuff structure 6 is not limited to the flow path body 92.

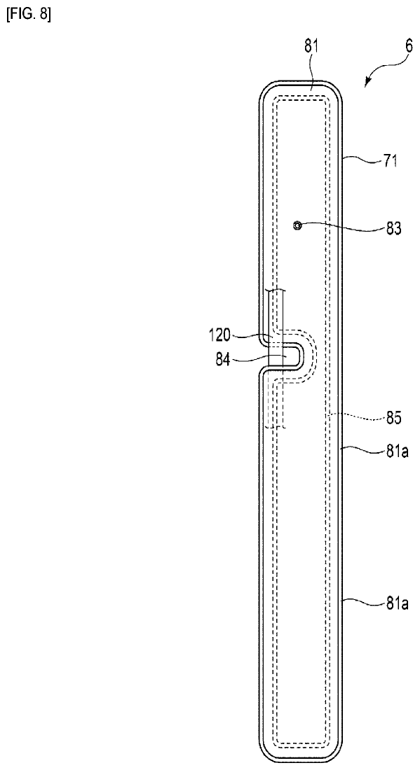

In another example, as in a modification illustrated in FIG. 8, electric wiring 120 for electrically connecting the device body 3 and the cuff structure 6 may pass through the cutout portion 84. FIG. 8 illustrates a part of the electric wiring 120.

As an example of the configuration in which the electric wiring 120 passes through the cutout portion 84, there is a configuration in which electrical components used for blood pressure measurement, for example, the pump 14 and the pressure sensor 15 are installed at a position proximate to the sensing cuff 73, and the electrical components are electrically connected to the control board 16 in the device body 3 by the electric wiring 120. The control board 16 in the device body 3 controls the pump 14 via, for example, the electric wiring 120. The pressure sensor 15 transmits a detection result to the control board 16 via, for example, the electric wiring 120. The electric wiring 120 is, for example, a flexible printed wiring board.

In this manner, by passing the electric wiring 120 through the cutout portion 84, it is possible to electrically connect the electrical component disposed proximate to the living body with respect to the pressing cuff 71 to the control board 16 in the device body 3. Furthermore, by disposing the pump 14 and the pressure sensor 15 at a position proximate to the cuff structure 6, the device body 3 can be miniaturized.

Furthermore, as another example of the electrical component disposed proximate to the cuff structure 6, an electrocardiogram measurement electrode may be provided at a position in contact with the wrist 100 in the cuff structure 6, and the electrode and the control board 16 in the device body 3 may be electrically connected to each other by the electric wiring 120. According to this configuration, an electrocardiogram can be measured by the blood pressure measurement device 1. The pump 14, the pressure sensor 15, and the electrocardiogram measurement electrode may be provided in the cuff structure 6 and may be connected to the control board 16 of the device body 3 by the electric wiring 120. In addition, both the flow path body 92 and the electric wiring 120 may pass through the cutout portion 84.

That is, the present invention is not limited to the above-described embodiments, and various modifications can be made in an implementation stage without departing from the gist thereof. Furthermore, each of the embodiments may be carried out as appropriate in a combination as much as possible, and combined effects can be obtained in such a case. Furthermore, the inventions at various stages are included in the above-described embodiments, and the various inventions can be extracted in accordance with appropriate combinations in the plurality of disclosed constituent elements.

### REFERENCE NUMERALS LIST

1 Blood pressure measurement device
3 Device body
4 Belt
5 Curler
6 Cuff structure
11 Case
12 Display unit
13 Operation portion
14 Pump
15 Pressure sensor
16 Control board
31 Outer case
31a Lug
31b Spring rod
32 Windshield
35 Back lid
41 Button
43 Touch panel
61 First belt
61a First hole portion
61c Buckle
61d Frame body
61e Prong
62 Second belt
62a Small hole
71 Pressing cuff
72 Back plate
73 Sensing cuff
81 Air bag
81a First fixing portion
83 Connection portion
84 Cutout portion
85 Second fixing portion
86 Sheet member
86a First sheet member
86a1 Outer surface
86b Second sheet member
86c Third sheet member
86d Fourth sheet member
86e Fifth sheet member
86f Sixth sheet member
87 Opening
87b Opening
87c Opening
87d Opening
87e Opening
91 Air bag
92 Flow path body
93 Connection portion
96 Sheet member
96a Seventh sheet member
96b Eighth sheet member
100 Wrist
110 Artery
120 Electric wiring

What is claimed is:

1. A cuff comprising:
a plurality of bag-like structures layered with longitudinal directions aligned windably around a living body, each of the plurality of bag-like structures being configured by two sheet members long in one direction being fixed to each other at outer circumferential edges;
a cutout portion provided at the same position of each of the plurality of bag-like structures in the longitudinal direction, the cutout portion being cut out from one end toward the other end in a width direction of the bag-like structure; and
a fixing portion provided in a frame shape inside the outer circumferential edges of a sheet member of the two sheet members and a sheet member of the two sheet members facing each other of the corresponding two adjacent bag-like structures of the plurality of bag-like structures, the fixing portion being configured to fix the two adjacent bag-like structures to each other.

2. The cuff according to claim 1, wherein the cutout portion is formed avoiding a position facing an artery in a state of the cuff being wound around the living body.

3. The cuff according to claim 1, wherein a plurality of the cutout portions are provided.

4. A blood pressure measurement device comprising:
the cuff according to claim 1;
a sensing cuff pressed by the cuff;
a pump configured to supply fluid to the cuff and the sensing cuff;
a pressure sensor configured to detect a pressure in the cuff and the sensing cuff; and
a body attached to the cuff and the sensing cuff and including a control board being built into the body.

5. The blood pressure measurement device according to claim 4, wherein the cutout portion is formed avoiding a position facing an artery in a state of the blood pressure measurement device being attached to the living body.

6. The blood pressure measurement device according to claim 4, wherein a plurality of the cutout portions are provided.

7. The blood pressure measurement device according to claim 4, further comprising a flow path fluidly communicating with the sensing cuff,
wherein the flow path passes through the cutout portion.

8. The blood pressure measurement device according to claim 4, further comprising electric wiring electrically connected to the control board,
wherein the electric wiring passes through the cutout portion.
